(19) United States
(12) Patent Application Publication
(10) Pub. No.: US 2010/0087920 A1
(43) Pub. Date: Apr. 8, 2010

(54) Corneal Onlay Lenses and Related Methods for Improving Vision of Presbyopic Patients

(75) Inventor: J. Christopher Marmo, Danville, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: FORSIGHT LABS, LLC, Menlo Park, CA (US)

(21) Appl. No.: 12/247,144

(22) Filed: Oct. 7, 2008

(51) Int. Cl. A61F 2/14; B29D 11/00

(52) U.S. Cl. 623/5.11; 264/1.1

(57) ABSTRACT

Methods for improving vision in a person diagnosed with presbyopia include placing a corneal onlay lens having a lens body diameter less than 5 mm in contact with an eye of the person. The corneal onlay lens has at least one refractive region having a refractive power to correct vision effects caused by the presbyopia. If the person is a presbyopic emmetrope, the method may include a step of placing only one corneal onlay lens in the person to treat presbyopia or improve the vision of the person. Additional corneal onlay lenses and methods of making such corneal onlay lenses are also described.

### CROSS-REFERENCES TO RELATED APPLICATIONS

NOT APPLICABLE

### BACKGROUND OF THE INVENTION

### Field of the Invention

The present invention relates to optical treatment methods and ocular prostheses and manufacture of ocular prostheses useful in the methods. In particular, methods and devices for correcting presbyopia or improving the vision of a person diagnosed with presbyopia are described.

A person's vision can be corrected or improved by a variety of methods such as by wearing spectacles, wearing contact lenses, or even by surgery. In addition, ocular implants such as corneal onlays and corneal inlays have been described as being suitable for correcting a person's vision. Some ocular implants are blanks that do not have refractive power. In comparison, a corneal onlay refers to a lens having a refractive power and the lens provides vision correction to an eye when the lens is placed between the corneal epithelium of an eye and Bowman's membrane of the eye. A corneal inlay refers to a lens that provides vision correction when the lens is implanted behind Bowman's membrane in the stroma of the cornea of an eye.

Presbyopia is a condition in which a person loses the ability over time or has a decreased ability to focus clearly on nearby objects. It is believed that presbyopia may be caused, at least in part, by a decreased accommodation of the lens in the patient's eye. Presbyopia is most frequently diagnosed in people who are about forty years old or older. A patient with normal vision or who has emmetropic eyes and who is diagnosed with presbyopia is referred to as a presbyopic emmetrope. A patient who does not have normal vision or has ametrop eyes and who is diagnosed with presbyopia is referred to as a presbyopic ametrope. A presbyopic ametrope that is nearsighted is referred to as a presbyopic myope since the patient's eyes are myopic. A presbyopic ametrope that is farsighted is referred to as a presbyopic hyperope since the patient's eyes are hyperopic.

### BRIEF SUMMARY OF THE INVENTION

In one aspect, methods of improving or correcting vision of a person are provided. For example, methods of improving vision of a presbyopic person include placing a refractive corneal onlay lens in contact with an eye of a presbyopic person. The corneal onlay lens has an anterior surface and an opposing posterior surface. The corneal onlay lens is placed in the person's eye so that the corneal epithelium of the eye covers an anterior surface of the lens and a posterior surface of the lens contacts Bowman's membrane of the eye. The corneal onlay lens has a refractive region having a refractive power for improving the vision of the eye of the person. The corneal onlay lens has a lens body diameter less than 5 mm.

In another aspect, corneal onlay lenses that include lens bodies that improve or correct the vision of a presbyopic person are provided. For example, a corneal onlay lens includes a lens body having a corneal epithelium contactable anterior surface, an opposing Bowman's membrane contactable posterior surface, a lens body diameter less than 5 mm, and a refractive region having a refractive power for improving the vision of an eye of the presbyopic person. The corneal epithelium contactable anterior surface will be in contact with the corneal epithelium of a person's eye when the lens is placed in contact with the eye. The Bowman's membrane contactable surface will be in contact with Bowman's membrane of a person's eye when the lens is placed in contact with the eye.

In yet another aspect, methods of producing corneal onlay lenses that improve or correct a presbyopic person's vision are provided. For example, a method of producing a corneal onlay includes shaping a lens forming material into a lens body having a corneal epithelium contactable anterior surface, an opposing Bowman's membrane contactable posterior surface, a lens body diameter less than 5 mm, and at least one refractive region having a refractive power for improving the vision of an eye of the person.

Uses of corneal onlay lenses are also described herein.

Various embodiments of the present invention are described in detail in the detailed description and additional disclosure below. Any feature or combination of features described herein are included within the scope of the present invention provided that the features included in any such combination are not mutually inconsistent as will be apparent from the context, this specification, and the knowledge of one of ordinary skill in the art. In addition, any feature or combination of features may be specifically excluded from any embodiment of the present invention. Additional advantages and aspects of the present invention are apparent in the following detailed description, drawings, and additional disclosure.

### BRIEF DESCRIPTION OF THE DRAWINGS

FIG. 1 is a flow chart of a method of correcting presbyopia or for improving vision effects caused by presbyopia;

FIG. 2 is an illustration of a corneal onlay lens for correcting presbyopia;

FIGS. 3A and 3B illustrate placement of a corneal onlay lens on an eye in which the corneal epithelium has been removed;

FIGS. 4A, 4B, and 4C illustrate placement of a corneal onlay lens on an eye in which the lens is covered by a corneal epithelial flap; and

FIGS. 5A, 5B, and 5C illustrate placement of a corneal onlay lens on an eye in which the lens is inserted into a corneal epithelial pocket.

### DETAILED DESCRIPTION OF THE INVENTION

The present methods of correcting or improving vision of a person or patient include placing a corneal onlay lens in contact with Bowman's membrane of a deepithelialized region of the patient's eye such that after a healing time period, a refractive lens is located between the corneal epithelium and Bowman's membrane.

In one aspect, methods for correcting presbyopia of a person are provided. The methods can be understood to also be methods of improving vision of a person diagnosed with presbyopia or methods of improving vision of a presbyopic person. Presbyopia refers to a condition in which a person loses the ability over time, or has a decreased ability, to focus clearly on nearby objects, and it may be caused by a decreased accommodation of the lens in the patient's eye. The presbyopic person or people (e.g., presbyopes) that can benefit from the present methods and devices are typically older the about 40 years old. For example, presbyopes ranging in age from about 45 years old to about 70 years old can benefit from the present methods and devices. A person is diagnosed as a presbyope by a skilled professional using conventional eye examination techniques that, among other things, are used to evaluate accommodation of the person's eyes. However, some early signs of presbyopia may be apparent to the patient without the eye exams, such as the decreasing ability to focus on near objects while still being able to focus on distant objects.

The present methods are directed at improving the vision of presbyopes. The present methods are particularly useful in improving the vision of presbyopic emmetrope. For example, a presbyopic emmetrope is a person with normal vision other than the decreased accommodation and resulting loss of clear near vision. A presbyopic emmetrope does not require any additional myopic or hyperopic vision correction. In comparison, a presbyopic ametrope is a person who is myopic or hyperopic, respectively, who also is presbyopic.

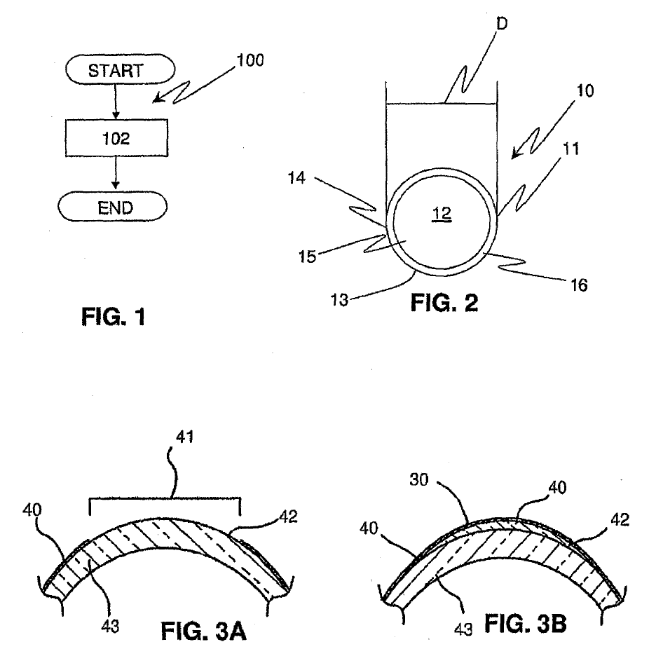

A method 100 for improving the vision of a presbyopic person is illustrated in FIG. 1. The method 100 includes a step 102 of placing the corneal onlay lens in contact with an eye of a presbyopic person. The corneal onlay lens is placed in contact with the eye so that the corneal epithelium of the eye covers an anterior surface of the corneal onlay lens, and so that a posterior surface of the corneal onlay lens contacts Bowman's membrane of the eye. The corneal onlay lens has a refractive region having a refractive power for improving the vision of the eye of the presbyopic person. The method 100 may also include one or more additional steps as discussed herein.

As shown in FIG. 2, a corneal onlay lens 10 has a lens body 11. The lens body 11 has an anterior surface 12, an opposing posterior surface 1'3, and a lens edge 14 at the periphery of the lens body 11. The lens body 11 also has a lens body diameter D. The embodiment of the corneal onlay lens 10 illustrated in FIG. 2 has a refractive region or central area 15 having a refractive power. The central area 15 may also be understood to be a vision correcting zone or an optic zone. Usually, the center of the central area 15 will include the optical axis of the lens. A ramp zone 16 is located between the lens edge 14 and the central area 15. The ramp zone 16 can be understood to be region of the lens body 11 where the thickness of the lens body increases from about 0 micrometers at the lens edge 14 to the thickness of the outer periphery of the central area 15. Typically, the ramp zone 16 does not provide substantial or any vision correction.

The refractive region of he present corneal onlay lenses have a desired refractive power to improve the patient's vision based on the eye examination information of the patient. In situations where the patient is a presbyopic emmetrope, the corneal onlay lens has a refractive region that has a refractive power to provide clear near vision to the person.

In embodiments of the corneal onlay lens 10 illustrated in FIG. 2, or other similarly designed corneal onlay lenses, the central are 15 or optic zone can have a diameter from about 0.5 mm to about 4 mm. The corneal onlay lenses can have the lens body diameters from about 1.0 mm to about 4.5 mm. The refractive power of the central area 15 can range from about +0.75 diopters to about +3.0 diopters. One embodiment of a corneal onlay lens includes a lens body having a lens body diameter of 1 mm and an optic zone diameter of 0.5 mm. Another embodiment of a corneal onlay lens includes a lens body having a lens body diameter of 4.5 mm and an optic zone diameter of 4 mm. The optic power of the optic zone can vary among lenses, and can have a power of any value between +0.75 diopters to +3.0 diopters. Typically, different powers can be provided among corneal onlay lenses in 0.25 diopter increments.

FIG. 3A and FIG. 3B illustrate one embodiment of the present methods. As shown in FIG. 3A, the corneal epithelium 40 is removed or separated from the corneal Bowman's membrane 42 and corneal stroma 43 to create a deepithelialized region 41 of a person's eye. The deepithelialized region 41 can be created by mechanically removing the corneal epithelium or chemically removing the corneal epithelium. Once the corneal epithelium 40 has been removed, a corneal onlay lens 30, such as the corneal onlay lens 10 described above, is placed in contact with Bowman's membrane 42, as shown in FIG. 3B. The corneal epithelium 40 is allowed to grow over the corneal onlay lens 30 such that the lens is located between the corneal epithelium 40 and Bowman's membrane 42.

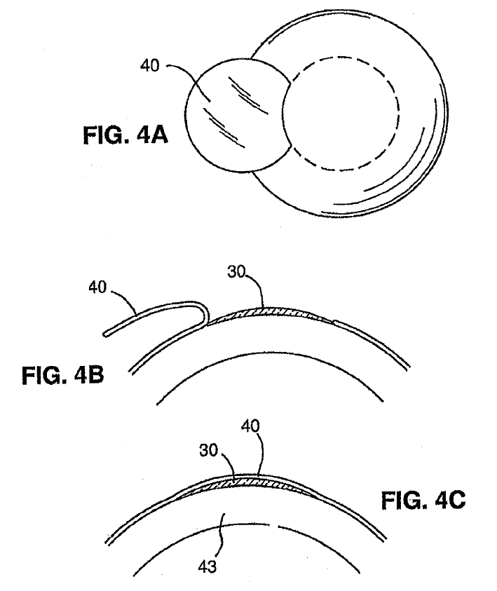

FIG. 4A illustrates the formation of a flap of corneal epithelium 40 that has been cut and separated from Bowman's membrane. The flap can be formed by using a microkeratome or other mechanical device to mechanically delaminate the corneal epithelium from the Bowman's membrane. As shown in FIG. 4b, with the flap or corneal epithelium 40 separated from the Bowman's membrane, a corneal onlay lens 30, such as the corneal onlay lens 10, is placed in contact with the Bowman's membrane. The flap of corneal epithelium 40 is then placed over the corneal onlay lens 30, as shown in FIG. 4C so that the corneal onlay lens is located between the epithelium and Bowman's membrane.

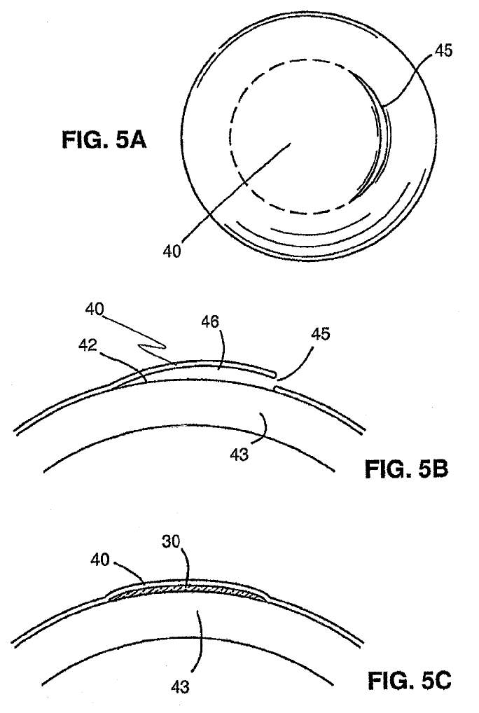

Another embodiment of the present methods is illustrated in FIGS. 5A, 5B, and 5C. In FIG. 5A, an incision 45 is formed in the corneal epithelium 40. The incision is formed laterally and paced apart from the central optic axis of the eye. A corneal epithelial pocket 46 or cavity is formed between the corneal epithelium 40 and Bowman's membrane 42 (FIG. 5B). A corneal onlay lens 30 is placed in the pocket 46 through incision 45 so that the corneal onlay lens is located between the epithelium and Bowman's membrane.

Thus, embodiments of the present methods can include steps such as separating the corneal epithelium from Bowman's membrane of the eye and placing the corneal onlay lens on the deepithelialized region of the cornea, which in certain embodiments, can include placing the corneal onlay lens in a corneal epithelial pocket or covering the corneal onlay lens located on Bowman's membrane with a flap of corneal epithelium.

In addition, it can be understood that in certain embodiments, the method comprises or consists essentially of placing only one corneal onlay lens in only one eye of the person to improve the vision of the person. Thus, instead of placing two corneal onlay lenses in the patient's eyes (i.e., one in each eye), embodiments of the present methods improve a presbyopic person's vision by placing only one lens in one eye. For example, presbyopic patients typically have a dominant eye and a non-dominant eye. The dominant eye is predominant for distance vision, and the other eye is the non-dominant eye. Thus, in methods in which the corneal onlay lens is structured to provide near vision correction, the methods can comprise a step of placing the one corneal onlay lens in the non-dominant eye of the person.

In addition, in view of the foregoing description, it can be appreciated that certain embodiments of the present methods may comprise a step of forming an incision in the corneal epithelium. For example, in the formation of a corneal epithelial pocket, an incision can be formed through which the corneal onlay passes when the lens is placed in contact with the eye. The width of the incision should be relatively small and at the greatest, should correspond to the lens body diameter of the corneal onlay lens. When a corneal onlay lens has a lens body diameter less than 5 mm, the width of the incision should be 6 mm or less. When corneal epithelial pockets are formed, the corneal epithelial pocket is dimensioned, such as sized and shaped, to retain the corneal onlay lens in the pocket without the lens being extruded from the pocket, especially during the healing of the incision in the corneal epithelium. In comparison, when corneal onlay lenses having a lens body diameter greater than 5 mm are inserted in corneal epithelial pockets, it has been observed that during the healing of the epithelium (e.g., within a few days after the surgical procedure), the corneal onlay lens can be extruded from the pocket. The present methods are able to improve the vision of a presbyopic patient by providing near vision, even despite the relatively small size of he corneal onlay lens. Compared to methods in which a corneal onlay lens having a lens body diameter greater than 5 mm is inserted into a corneal epithelial pocket, the present methods provide for reduced healing times and improved healing processes since the incisions are relatively smaller, the disruption of the corneal epithelium is less, and reattachment of the corneal epithelium over the lens is improved.

Furthermore, it can be appreciated that embodiments of the present methods may comprise a step of separating a portion of the corneal epithelium from the Bowman's membrane before placing the corneal onlay lens in contact with the eye. For example, a portion of the patient's corneal epithelium can be mechanically or chemically removed from the eye to produce a deepithelialized region of the eye, or a layer of corneal epithelium can be separated from the Bowman's membrane to form a corneal epithelial flap, as discussed herein.

In the separation of the corneal epithelium from the Bowman's membrane, including when corneal epithelial pockets are being formed, the separation of the corneal epithelium may comprise delaminating the portion of the corneal epithelium from the Bowman's membrane using a microkeratome. For example, a suction ring device can be placed in contact with a patient's eye, and a microkeratome blade can move relative to the suction ring and mechanically separate a portion of the corneal epithelium from the Bowman's membrane.

The present methods may also include additional steps, such as cooling the corneal epithelium or eye, applying a liquid composition, including saline, to the eye, administering an anesthetic to the person, or combinations thereof.

In another aspect, corneal onlays are provided and described. The present corneal onlays include a lens body having a corneal epithelium contactable anterior surface, an apposing Bowman's membrane contactable posterior surface, a lens body diameter less than 5 mm, and a refractive region having a refractive power for improving the vision of a presbyopic person when the corneal onlay lens is located between the corneal epithelium and Bowman's membrane of the eye of the person.

In certain embodiments, the corneal onlay lens has a lens body diameter from about 1 mm to about 4.5 mm. The corneal onlay can have a central area that has a refractive power to correct presbyopia of the person's eye. An example of such corneal onlays are illustrated in FIG. 2.

The refractive region of the present corneal onlays can have a refractive power from about +0.75 diopters to about +3.0 diopters. In addition, the refractive region can have other refractive powers to provide near vision in the patient's eye. A corneal onlay in accordance with the present disclosure may have only one refractive region or optic zone, and the diameter of the refractive region can be from about 0.5 mm to about 4.0 mm. For example, one corneal onlay lens that can treat presbyopia can have a lens body diameter of 1.0 mm and an optic zone diameter of about 0.5 mm. In another example, a corneal onlay lens that can correct presbyopia can have a lens body diameter of 4.5 mm and an optic zone diameter of 4.0 mm.

In certain embodiments of the present onlays, the optic zone diameter and the lens body diameter are equal. For example, in such embodiments, the lens body would not include a ramp zone or peripheral zone. In embodiments in which the optic zone of the lens extends to the peripheral edge of the lens, the curvature of the anterior surface or posterior surface of the lens extending from the center of the lens to the edge (e.g., a radial length) is defined by a single spherical curve or a single aspherical curve. In comparison, when a ramp zone is a portion of the lens body, a visibly identifiable junction is present that would prevent the radial length from being accurately described with a single spherical curve or a single aspherical curve.

In another aspect, methods of producing corneal onlays are provided. The production methods include a step of shaping a lens forming material into a lens body having a corneal epithelium contactable anterior surface, an opposing Bowman's membrane contactable posterior surface, a lens body diameter less than 5 mm, and at least one refractive region having a refractive power for improving the vision of an eye of a presbyopic person when the corneal onlay lens is located between the corneal epithelium and Bowman's membrane of the eye of the person.

The shaping step may include molding a lens forming material, lathing a lens forming material, or combinations thereof, to form the lens body.

The lens forming material can be understood to be a polymerizable formulation containing reactive ingredients. In certain embodiments, the polymerizable formulation includes collagen, such as recombinant collagen, that can be crosslinked with a crosslinking agent during a polymerization process. Such polymerizable formulations can be placed in a mold to polymerize the formulation to form a polymerized product. In certain embodiments, the polymerized product is in the shape of a lens having a refractive power. In further embodiments, the polymerized product is a cast molded lens that is formed in corneal onlay mold assembly including a front surface mold having a concave lens forming surface and a back surface mold having a convex lens forming surface, wherein the front surface mold and back surface mold are assembled together to form a lens shaped cavity.

The lens forming material may also be understood to be a polymerized product. The polymerized product can be a lens shaped article obtained from a corneal onlay lens mold assembly. Alternatively, the polymerized product can be a rod shaped article obtained from a cylindrical mold. The polymerized product may be lathed, machined, or otherwise ablated to form a corneal onlay lens, as described herein.

The final corneal onlay lens should be formed of a material that is biocompatible and provides sufficient nutrient and gas exchange to maintain a viable corneal epithelium and still provide the desired refractive correction.

In certain embodiments, the shaping comprises shaping the lens forming material into a lens body having a diameter from about 1 mm to about 4.5 mm. In addition to alternatively, the shaping may include shaping the lens forming material into a lens body having only one refractive region, and the diameter of the refractive region is from about 0.5 mm to about 4.0 mm.

In view of the disclosure here, it can also be understood that an additional aspect of the present invention relates to the use of a corneal onlay to correct vision effects caused by presbyopia, such as by using any of the corneal onlay lens embodiments described herein to correct vision effects caused by presbyopia.

In yet a further additional aspect, the present invention includes the use of a lens forming material in the manufacture of a corneal onlay lens for correcting vision effects caused by presbyopia, such as by using a lens forming material to manufacture any of the corneal onlay lens embodiments described herein.

Although the disclosure herein refers to certain specific embodiments, it is to be understood that these embodiments are presented by way of example and not by way of limitation. The intent of the foregoing detailed description, although discussing exemplary embodiments, is to be construed to cover all modifications, alternatives, and equivalents of the embodiments as may fall within the spirit and scope of the invention as defined by the claims.

What is claimed is:

1. A method for improving vision of a presbyopic person, the method comprising:
placing a corneal onlay lens in contact with an eye of a presbyopic person, the eye having a corneal epithelium, such that the corneal epithelium of the eye covers an anterior surface of the lens and a posterior surface of the lens contacts Bowman's membrane of the ye, the corneal onlay lens having a lens body diameter less than 5 mm and structure to improve vision of the presbyopic person.

2. The method of claim 1 wherein the method comprises placing only one corneal onlay lens in only one eye of the person to improve the vision of the person.

3. The method of claim 1 or 2, wherein the lens has a refractive region having a refractive power to provide clear near vision to the person.

4. The method of claim 3, wherein the lens body diameter is from about 1 mm to about 4.5 mm.

5. The method of claim 4, wherein the corneal onlay lens has only one refractive region, and the diameter of the refractive region is from about 0.5 mm to about 4.0 mm.

6. The method of claim 5, wherein the refractive region has a refractive power from about +0.75 diopters to about +3.0 diopters.

7. The method of claim 2, wherein he person has a dominant eye and a non-dominant eye, and the placing comprises placing the one corneal onlay lens in the non-dominant eye.

8. The method of claim 1, further comprising forming an incision in the corneal epithelium through which the corneal onlay lens passes when the corneal onlay lens is placed in contact with the eye.

9. The method of claim 8, wherein the incision has a width no greater than 6 mm.

10. The method of claim 1, further comprising separating a portion of the corneal epithelium from the Bowman's membrane before placing the corneal onlay lens in contact with the eye.

11. The method of claim 10, wherein the separating comprises delaminating the portion of the corneal epithelium from the Bowman's membrane using a microkeratome.

12. The method of claim 1, further comprising forming a corneal epithelial pocket to accommodate the corneal onlay lens, the corneal epithelial pocket being dimensioned to retain the corneal onlay therein without the corneal onlay lens being extruded therefrom.

13. A corneal onlay, comprising:
a lens body having a corneal epithelium contactable anterior surface, an opposing Bowman's membrane contactable posterior surface, a lens body diameter less than 5 mm, and a refractive region having a refractive power for improving the vision of an eye of a presbyopic person when he corneal onlay lens is located between the corneal epithelium and Bowman's membrane of the eye of the presbyopic person.

14. The corneal onlay of claim 13, wherein the lens body has a diameter from about 1 mm to about 4.5 mm.

15. The corneal onlay of claim 13, wherein the corneal onlay lens has only one refractive region, and the diameter of the refractive region is from about 0.5 mm to about 4.0 mm.

16. The corneal onlay of claim 13, wherein the refractive region has a refractive power from about +0.75 diopters to about +3.0 diopters.

17. A method of producing a corneal onlay, comprising:
shaping a lens forming material into a lens body having a corneal epithelium contactable anterior surface, an opposing Bowman's membrane contactable posterior surface, a lens body diameter less than 5 mm, and at least one refractive region having a refractive power for improving the vision of an eye of a presbyopic person when the corneal onlay lens is located between the corneal epithelium and Bowman's membrane of the eye.

18. The method of claim 17, wherein the shaping comprises molding a lens forming material, lathing a lens forming material, or combinations thereof, to form the lens body.

19. The method of claim 17, wherein the shaping comprises shaping the lens forming material into a lens body having a diameter from about 1 mm to about 4.5 mm.

20. The method of claim 17, wherein the shaping comprises shaping the lens forming material into a lens body having only one refractive region, and the diameter of the refractive region is from about 0.5 mm to about 4.0 mm.

21. Use of a corneal onlay lens to correct vision effects caused by presbyopia.

22. Use of a lens forming material in the manufacture of a corneal onlay lens for correcting vision effects caused by presbyopia.
